(19) United States
(12) Patent Application Publication
(10) Pub. No.: US 2026/0327265 A1
(43) Pub. Date: Oct. 22, 2026

(54) NOVEL ACETYLATED PEPTIDES WITH GCMAF-LIKE ACTIVITIES, AND METHODS FOR THEIR SYNTHESIS

(71) Applicant: Marco Ruggiero, Mesa, AZ (US)

(72) Inventor: Marco Ruggiero, Mesa, AZ (US)

(21) Appl. No.: 19/562,664

(22) Filed: Mar. 10, 2026

Related U.S. Application Data
(60) Provisional application No. 63/788,083, filed on Apr. 13, 2025.

(51) Int. Cl. C07K 7/08; C07K 1/04

(52) U.S. Cl. CPC C07K 7/08; C07K 1/042

(57) ABSTRACT

Disclosed are novel acetylated peptides for immune modulation and methods for their synthesis. Specifically, two distinct acetylated amino acid sequences are provided. These sequences exhibit GcMAF-like activity, demonstrating capacity for macrophage activation and potential anti-cancer activity. These defined synthetic peptides represent potential tools or therapeutic agents.

### GOVERNMENT CONTRACT

Not applicable

### CROSS-REFERENCE TO RELATED U.S. APPLICATIONS DATA

This application claims the benefit of U.S. Provisional Patent Application No. 63/788,083. Patent Center No. 69983606. Confirmation No. 2465. Filed on Apr. 13, 2025.

### STATEMENT RE. FEDERALLY SPONSORED RESEARCH/DEVELOPMENT

Not applicable

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The technical field relates to immunomodulatory compounds, specifically peptides exhibiting macrophage activating factor-like activity, and methods for their synthesis.

### REFERENCES CITED

### U.S. Patent Documents

Method for producing activated GcMAF
   JP6718561B2 Foundation for Biomedical Research and Innovation at Kobe
   Filed 2018 Dec. 14. Published 2020 Jul. 8. Granted 2020 Jul. 8
Macrophage activating factor for pharmaceutical compositions
   WO EP US CN JP AU CA US20170173115A1 Nobuto Yamamoto
   Priority 2011 Apr. 7. Filed 2017 Feb. 27. Published 2017 Jun. 22
Vitamin d complexes with de-vdbp and an unsaturated fatty acid, and their use . . .
   WO EP US JP RU US20160220587A1 Marco Ruggiero
   Priority 2013 Jun. 21. Filed 2014 Jun. 13 ·Published 2016 Aug. 4

### OTHER PUBLICATIONS

Nabeshima, Y., Abe, C., Kawauchi, T. et al. Simple method for large-scale production of macrophage activating factor GcMAF. Sci Rep 10, 19122(2020 ). https://doi.org/10.1038/s41598-020-75571-y
Gregory K J, Zhao B, Bielenberg D R, Dridi S, Wu J, Jiang W, Huang B, Pirie-Shepherd S, Fannon M. Vitamin D binding protein-macrophage activating factor directly inhibits proliferation, migration, and uPAR expression of prostate cancer cells. PLOS One. 2010 Oct. 18; 5(10): e 13428. doi: 10.1371/journal. pone.0013428. PMID: 20976141; PMCID: PMC2956649.
Ruggiero M. (2024a) GcMAF in radiation therapy: identification and molecular characterization of the human GcMAF receptor. Int J Radiol Radiat Ther. 11(3): 77-80. DOI: 10.15406/ijrrt.2024.11.00390.
Ruggiero, M. (2024b). Genetic Analysis of Bifidobacteriaceae in a Fermented Milk and Colostrum Product in Relation to Immune Function. American Journal of Immunology, 19(1), 24-34. https://doi.org/10.3844/ajisp.2024.24.34.

### INVENTOR'S EXPERTISE

Dr. Marco Ruggiero, MD, PHD, has accrued substantial research experience in the area of immune-modulating and anti-cancer molecules such as GcMAF. This expertise is described in the profile reported below.

The evolution of scientific thought documented in this profile did not emerge from an academic vacuum; rather, it is rooted in a robust generational continuity dedicated to observational medicine and clinical excellence. Born in Florence, Italy, in 1956, Dr. Ruggiero hails from a family of physicians: both parents were surgeons who held dual specializations in Radiology and Radiotherapy, as well as in Dentistry and Dental Prosthetics. A constant touchstone in this intellectual heritage is represented by the clinical observations made in 1908 by Dr. Ruggiero's grandfather, a physician who, while documenting the phenomenology of measles, intuited dynamics of heterologous immunity that modern science would only validate a century later. [1]. This imprinting defined a method: the primacy of empirical clinical data over preconceived theory.

Dr. Ruggiero's academic path followed this vocation with rigor: he graduated in Medicine and Surgery from the University of Florence at the age of 24 with the highest honors (summa cum laude) and subsequently earned a PhD in Molecular Biology, consolidating the technical foundations for his future avant-garde research. Significant to his human and professional development was his experience as a Medical Officer in the Italian Army, operating within NATO forces-an assignment that added a dimension of discipline and service to his vision of medical practice.

His formal scientific debut occurred in 1981, with research dedicated to the nutritional status and dietary conditions in tropical countries [2]. Although seemingly distant from the molecular biology that would characterize subsequent decades, this work laid the groundwork for a thermodynamic vision of the human organism and its environmental interactions. Within that context, nutrition was analyzed not merely as caloric intake, but as a flow of energy within an open system. The concepts of Primary Energy (E1)—energy captured from the environment—and Secondary Energy (E2)—energy generated by human activity—were already present in that 1981 study and have recently been revisited by Dr. Ruggiero to perform a systematic comparison between historical and contemporary data, analyzing the evolution of energy transformation processes within biological systems [3]. The circle begun in 1981 finds its completion today in a synthesis that views biology not as inert matter, but as an incessant transformation of energetic signals.

The necessity of understanding how primary energy was translated into cellular decisions led Dr. Ruggiero to the United States, the epicenter of the 1980s biotechnological revolution. A pivotal milestone was represented by his collaboration with Eduardo G. Lapetina [4] at the Burroughs Wellcome Research Laboratories (North Carolina).

A turning point in the scientific output of those years was the 1986 publication in the Proceedings of the National Academy of Sciences (PNAS), a paper presented by Nobel Laureate Sir John Vane titled “Protease and cyclooxygenase inhibitors synergistically prevent activation of human platelets.” In this study [5], Dr. Ruggiero, alongside Eduardo G. Lapetina, described a potent synergistic effect between the protease inhibitor leupeptin and the cyclooxygenase inhibitor indomethacin in preventing platelet activation. The results showed that leupeptin alone only partially inhibited thrombin-induced aggregation, and indomethacin alone was ineffective against this agonist. However, their combination completely blocked both aggregation and serotonin release, while fibrin clot formation remained unaffected.

This discovery provided a mechanistic explanation for the often equivocal results found in clinical trials conducted with cyclooxygenase inhibitors alone, such as aspirin, highlighting how thrombin-mediated platelet activation represents a critical signaling pathway that can only be neutralized through a combined approach. The implications of this work remain highly relevant for the prophylaxis of thromboembolic diseases such as stroke and myocardial infarction, suggesting strategies to optimize antiplatelet therapies and reduce side effects through a significant reduction in dosages made possible by pharmacological synergism.

The journey continued at the National Cancer Institute (NCI) of the NIH, in the Laboratory of Cellular and Molecular Biology (LCMB) directed by Stuart A. Aaronson.

Bethesda at that time represented the world's pinnacle for oncogene research. In collaboration with Aaronson, Dr. Ruggiero contributed to the characterization of the Epidermal Growth Factor (EGF) receptor signaling cascade. The results of these studies, published in leading journals such as Science [6], PNAS [7,8], and Oncogene [9], demonstrated how the activation of specific oncogenes induces chronic stimulation of Phospholipase C, bypassing cellular control mechanisms. This phase allowed Dr. Ruggiero to map the molecular “circuits” of cancerous cells, acquiring the technical toolkit necessary for subsequent clinical challenges.

Upon his return to Italy, Dr. Ruggiero embarked on a distinguished academic career at the University of Florence [10], serving first as Associate Professor and subsequently as Full Professor. Holding the chair of Molecular Biology in the Faculty of Mathematical, Physical, and Natural Sciences, his courses were noted for an exceptionally high pedagogical impact.

According to the University's official “Evaluation of Teaching” system, the Molecular Biology course consistently ranked at the top of the Faculty, achieving a score of 8.94/10 and a student satisfaction rate of 98.2% in 2012-2013. Similar results were achieved at the Faculty of Engineering, where the course on “Advanced Methodologies in Medicine” reached a 100% satisfaction rating.

The scientific prestige of this period is attested by numerous honors displayed on the official homepage of the University of Florence. Of particular note was the resonance given in 2009 to research on AIDS pathogenesis which, through a critical analysis of ministerial epidemiological data, opened new fronts for methodological discussion [11,12]. Further mentions concerned the presentation at the XVIII International AIDS Conference in Vienna (2010) of studies on personalized immune responses based on VDR gene polymorphisms [13], as well as the publication of discoveries regarding the mechanism of action of GcMAF (Gc protein-derived Macrophage Activating Factor/Vitamin D binding protein Macrophage Activating Factor) in inhibiting the growth of tumor cells—works appearing in journals such as Cancer Immunology and Immunotherapy [14] and Anticancer Research [15]. Concurrently, his specialization in Radiodiagnostics obtained in Siena allowed him to integrate molecular rigor with clinical practice, focusing on the role of the Vitamin D Receptor (VDR) and the Vitamin D-Binding Protein (DBP) in modulating immunity and cellular radioresistance [16].

In the second decade of the 21st century, Dr. Ruggiero's career pivoted toward translational immunotherapy. The discovery and application of GcMAF was a watershed moment. By demonstrating how a specific modification of the Vitamin D-binding protein could activate macrophages to counter tumor progression and chronic viral infections, Dr. Ruggiero challenged dominant therapeutic paradigms [17].

This phase was characterized by a steadfast defense of scientific independence. Faced with the resistance of an often self-referential academic and industrial system, Dr. Ruggiero promoted the “Citizen Science” model. This approach advocates for transparent scientific research centered on patient needs and free from commercial bias, where the validity of a therapy is sanctioned by documented clinical results (“Actual Proof”).

In this context, Dr. Ruggiero embraced the role of inventor, translating laboratory discoveries into innovative and accessible nutritional solutions. His primary invention of this period is Bravo, a revolutionary product formulated to reconstitute microbiome diversity and endogenously produce immunomodulatory molecules. Bravo is not a mere probiotic, but a complex ecosystem [18] based on the fermentation of colostrum and milk (or plant-based proteins), where the coordinated action of hundreds of microbial strains and microbial-released enzymes allows for the natural synthesis of GcMAF and bioactive peptides [19].

The success of Bravo and the ongoing dialogue with the Citizen Science community prompted Dr. Ruggiero to extend the concept of biological protection to the damages caused by the modern electromagnetic environment [20]. From this innovative drive, the concept of Praesidium was born-conceived as an “internal shield” for cellular resilience. This intuition later culminated in a fully developed scientific concept and the award of a United States patent during his later years in Arizona.

This commitment led to the development of innovative protocols for complex pathologies such as autism, cancer, and certain chronic syndromes. An emblematic example of this integrative capacity is the formulation of the Ruggiero-Klinghardt (RK) Protocol [22], born from a collaboration with Dr. Dietrich Klinghardt. This protocol introduced a genuine paradigm shift in the diagnosis and therapy of chronic conditions—including persistent Lyme disease, neurodegenerative diseases, and oncology—through the synergistic use of diagnostic and therapeutic ultrasonography. By utilizing ultrasound as a “provocation” tool to render pathogens, toxins, and compartmentalized transformed cells “visible” to the immune system and laboratory methods, the RK Protocol optimizes drug uptake and treatment efficacy in specific targeted body areas.

His arrival in Arizona marked the beginning of a phase of profound synthesis in which molecular biochemistry merged with quantum physics, leading Dr. Ruggiero's research to challenge the boundaries of deterministic biology. At the core of this paradigm is the integration of the Orch OR (Orchestrated Objective Reduction) theory, proposed by Stuart Hameroff (University of Arizona, Tucson) and Nobel Laureate Sir Roger Penrose (University of Oxford, UK) [23,24]. The scientific robustness of Dr. Ruggiero s contributions in this field was attested by the presentation of his results at the 2022 and 2024 editions of the prestigious The Science of Consciousness (TSC) conference, held with the participation of the theory's founding fathers.

This dual expertise in traditionally distant fields such as immunology and radiology has found official recognition at the editorial level: Dr. Ruggiero serves as Editor-in-Chief for both the American Journal of Immunology (AJI) and the International Journal of Radiology and Radiation Therapy (IJRRT). This privileged position reflects the researcher's ability to integrate the microscopic analysis of molecular signals with the macroscopic vision of radiological imaging-a synthesis necessary for addressing complex subjects such as oncology, immunology, imaging science, and the science of consciousness.

In this context, a fundamental milestone is represented by the identification of the GcMAF receptor in human macrophages, published in IJRRT in 2024[25 ]. The discovery of the molecular “key” through which this protein transmits its signal provided definitive proof of GcMAF's mechanism of action, linking membrane biochemistry to the modulation of the cytoskeleton and intracellular informational processes.

Dr. Ruggiero's radiological expertise found its peak technological expression in the United States patent filed in 2021 and granted in 2024[21 ]. The invention concerns a revolutionary formulation to protect the organism from ionizing and non-ionizing radiation (including the 5G standard), based on biological quantum entanglement. The process utilizes Cherenkov radiation generated by natural isotopes to stimulate photosynthesis and induce DNA repair genes in commensal microorganisms, subsequently transferring this radio-resistance to human cells via the phage protein ORF252 [18]. This technology allows for the protection of healthy tissues during radiotherapy, maximizing therapeutic efficacy on neoplastic cells.

Within this journey, Dr. Ruggiero's human and spiritual dimension assumes fundamental relevance; he has practiced Nichiren Shoshu Buddhism since 1979. This discipline has influenced every aspect of his life, including his academic rigor and the direction of his research.

Two pioneering studies (2023-2024) examined the chanting of Nam-Myoho-Renge-Kyo. The first identified sound peaks at 8 Hz (Schumann resonance) and ‘solfeggio’ frequencies, which correlated with positive effects on the endocrine system and reduced oxidative stress.

Using fNIRS spectroscopy, a significant increase in prefrontal-cortex activity was observed, together with heightened metabolic activity in probiotic cultures [26]. The second study investigated a novel psychoneuroimmunological pathway, detecting consistent variations in natural radioactivity (counts per second, CPS) during chanting (measured with RadiaCode 10X) . These data offer what is defined in the Buddhist tradition as “Actual Proof,” suggesting a measurable interaction between mind, biology, and the environment.

Finally, in his 2024 work on the natural intelligence of the immune system [28], Dr. Ruggiero proposes that macrophages act as distributed quantum sensors. While the brain serves as a “reducing valve” for information (according to Aldous Huxley's famous definition), the immune system accesses the vast data of the microbiome, rendering consciousness a phenomenon interconnected with the energetic environment. Dr. Ruggiero's scientific path thus reaches an epistemological conclusion: the true understanding of life requires an act of intellectual freedom capable of uniting the precision of the molecule, the vision of the radiologist, and the depth of quantum physics, under the sole guidance of Reason.

The scientific path delineated herein—which moves from thermodynamic theses on nutrition in 1981 to the heights of quantum biology in 2026—does not represent a terminal point; rather, it documents the extraordinary synthesis achieved in Dr. Ruggiero's first seventy years of life. The transition from collaborations with Nobel laureates to the chair of Molecular Biology in Florence, through to the editorial leadership of prestigious journals and the recognition of his innovative genius via a U.S. patent, portrays a researcher and inventor whose trajectory is in a phase of full ascent.

This trajectory demonstrates that the understanding of complex systems cannot be divorced from a vision that unites the infinitesimal scale of quantum signaling with the macroscopic image of the human body. The integration of Orch OR theory with the study of the immune system's natural intelligence has found fundamental clinical application in the RK Protocol, which has revolutionized the approach to chronic pathologies, and in the invention of solutions such as Bravo and Praesidium.

Relying on the power of these innovations and the tangible results they generate in terms of vitality and resilience, Dr. Ruggiero—with a touch of intentional irony grounded in the “actual proof” of his own biology—does not consider his task completed in the slightest. On the contrary, he plans to continue his investigation into the mysteries of life and consciousness for the next seventy years, intending to maintain the same spirit of discovery that characterized his first seven decades.

A cornerstone of this perennial intellectual youth is the practice of Nichiren Shoshu Buddhism, which since 1979 has served as a catalyst for an unprejudiced scientific inquiry. The pursuit of “Actual Proof” constitutes the bridge between mysticism and quantum physics, proving that life is an interconnected system of energy and consciousness that knows no fixed chronological limits.

In an era of centralized knowledge, Dr. Ruggiero's legacy resides in the reaffirmation of Reason and independence. The “Citizen Science” model, combined with the ability to identify fundamental mechanisms such as the GcMAF receptor or biological entanglement, indicates that intellectual freedom is the only antidote to stagnation. Biology is not understood here as inert matter, but as a manifestation of consciousness itself—a field of inquiry that the author intends to explore for a long time to come.

While this profile focuses on the most significant milestones, the bibliography quoted in the EU Open Research Repository [29] constitutes the integral chronological collection of Dr.

Ruggiero's scientific production to date. It is conceived as an integral part of this evolving profile, offering an exhaustive overview of over forty years of commitment, and remaining open to the numerous publications that will follow in the decades ahead.

### REFERENCES

1. Ruggiero, M. (2025). Heterologous Immunity and Unknown Pandemics: A Physician's Observations From 1908 in Light of Modern Science. J Clin Exp Immunol, 10(2), 01-21.
2. Ruggiero, M. and Nicastro, C.: Considerazioni sulla situazione alimentare nei paesi tropicali e prospettive future. J. Agricult. Environ. for Intl. Dev. 75:435-450, 1981.
3. RUGGIERO, M., NICASTRO, C. THE E1/E2 PARADOX: TRILLIONS SPENT, 45 YEARS LATER, WHY IS THE Tropical Food System Functionally Worse?. Preprints 2025, 2025110993.
4. Watson, S. P., Ruggiero, M., Abrahams, S. and Lapetina, E. G.: Inositol 1,4,5-trisphosphate induces aggregation and release of 5-hydroxytriptamine from saponin permeabilized human platelets. J. Biol. Chem. 261:5368-5372, 1986.
5. Ruggiero, M. and Lapetina, E. G.: Protease and cyclooxygenase inhibitors synergistically prevent activation of human platelets. Proc. Natl. Acad. Sci. USA 83:3456-3459, 1986.
6. Pierce, J. H., Ruggiero, M., Fleming, T. P., Di Fiore, P. P., Greenberger, J. S., Varticovski, L., Schlessinger, J., Rovera, G., and Aaronson, S. A.: Signal transduction through the EGF receptor transfected into IL-3-dependent hematopoietic cells. Science 239:628-631, 1988.
7. Matsui, T., Pierce, J. H., Fleming, T. P., Greenberger, J. S., LaRochelle, W. J., Ruggiero, M. and Aaronson, S. A.: Independent expression of human alpha or beta PDGF receptor cDNAs in a naive hematopoietic cell leads to functional coupling with mitogenic and chemotactic signaling pathways. Proc. Natl. Acad. Sci. USA 86:8314-8318, 1989.
8. Pierce, J. H., Di Marco, E., Cox, G., Lombardi, D., Di Fiore, P. P., Ruggiero, M., Varesio, L., Sakaguchi, A. Y. and Aaronson, S. A.: CSF-1 induces proliferation, chemotaxis and reversible monocytic differentiation in myeloid progenitor cells transfected with the human c-fms/CSF-1 receptor cDNA. Proc. Natl. Acad. Sci. USA 87:5613-5617, 1990.
9. Ruggiero, M., Srivastava, S. K., Fleming, T. P., Ron, D., and Eva, A.: NIH/3 T3 fibroblasts transformed by the dbl oncogene show altered expression of bradykinin receptors. Effect on inositol lipid turnover. Oncogene 4:767-771, 1989.
10. Laffi, G., Marra, F., Failli, P., Ruggiero, M., Cecchi, E., Carloni, V., Giotti, A. and Gentilini, P.: Defective signal transduction in platelets from cirrhotics is associated with increased cyclic nucleotides. Gastroenterology 105:148-156, 1993.
11. Ruggiero M, Galletti M P, Pacini S, Punzi T, Morucci G, Gulisano M. On the risk of contracting AIDS at the dissection table. Ital J Anat Embryol. 2009 April-September; 114(2-3): 97-108. PMID: 20198822.
12. Duesberg P H, Mandrioli D, McCormack A, Nicholson J M, Rasnick D, Fiala C, Koehnlein C, Bauer H H, Ruggiero M. AIDS since 1984: no evidence for a new, viral epidemic—not even in Africa. Ital J Anat Embryol. 2011; 116(2): 73-92.
13. Thyer L, Ward E, Smith R, Fiore M G, Magherini S, Branca J J, Morucci G, Gulisano M, Ruggiero M, Pacini S. A novel role for a major component of the vitamin D axis: vitamin D binding protein-derived macrophage activating factor induces human breast cancer cell apoptosis through stimulation of macrophages. Nutrients. 2013 Jul. 8; 5(7): 2577-89. doi: 10.3390/nu5072577. PMID: 23857228; PMCID: PMC 3738989.
14. Pacini S, Morucci G, Punzi T, Gulisano M, Ruggiero M. Gc protein-derived macrophage-activating factor (GcMAF) stimulates cAMP formation in human mononuclear cells and inhibits angiogenesis in chick embryo chorionallantoic membrane assay. Cancer Immunol Immunother. 2011 April; 60(4): 479-85. doi: 10.1007/s 00262-010-0953-7. Epub 2010 Dec. 14. PMID: 21170647; PMCID: PMC11029590.
15. Ruggiero M, Ward E, Smith R, Branca J J, Noakes D, Morucci G, Taubmann M, Thyer L, Pacini S. Oleic Acid, deglycosylated vitamin D-binding protein, nitric oxide: a molecular triad made lethal to cancer. Anticancer Res. 2014 July; 34(7): 3569-78. PMID: 24982371.
16. Branca J J, Pacini S, Ruggiero M. Effects of Pre-surgical Vitamin D Supplementation and Ketogenic Diet in a Patient with Recurrent Breast Cancer. Anticancer Res. 2015 October; 35(10): 5525-32. PMID: 26408720.
17. Fiore M G, Magherini S, Gulisano M, Pacini S and Ruggiero M (2013). Study of aminoacidic sequences of vitamin D binding proteins involved in macrophage activation. Front. Immunol. Conference Abstract: 15th International Congress of Immunology (ICI). doi: 10.3389/conf.fimmu.2013.02.01124
18. Pacini S, Ruggiero M. Phage composition of a fermented milk and colostrum product assessed by microbiome array; putative role of open reading frames in reference to cell signaling and neurological development. J Neurol Stroke. 2020; 10(2): 80-90. DOI: 10.15406/jnsk.2020.10.00416
19. Carter, M., Pacini, S. & Ruggiero, M. (2020). Consumption of an Extremely Biodiverse Probiotic and a Supplement based on Microbial Chondroitin Sulfate is Associated with Very Low Serum Alpha-N-acetylgalactosaminidase (Nagalase) Activity and Decrease of C-reactive Protein Values. American Journal of Immunology, 16(1), 8-18.
20. Ruggiero, M. (2021). Experimental Evidence for the Role of Natural Radioactivity in Influencing Viability of Commensal Microorganisms. American Journal of Immunology, 17(1), 14-24.
21. Ruggiero M. (2021) Composition, methods and rationale for the formula of a pre-probiotic dietary supplement aimed at protecting the human body from the harmful effects of electromagnetic radiations through quantum entanglement of DNA repair genes elicited by natural superluminal particles inducing photosynthesis via Cherenkov effect. US Patent US20220401502A1
22. Klinghardt D and Ruggiero M. The Ruggiero-Klinghardt (RK) Protocol for the Diagnosis and Treatment of Chronic Conditions with Particular Focus on Lyme Disease. American Journal of Immunology 2017, 13(2 ): 114.126 DOI: 10.3844/ajisp.2017.114.126
23. Ruggiero M. (2021) The brain microbiota as it relates to the orch or theory of consciousness. J Neurol Stroke; 11(5): 131-133. doi: 10.15406/jnsk.2021.11.00471
24. Ruggiero M. (2023) Study of structural similarities between tubulin, TMC 1, AND FTSZ proteins as they relate to mechanosensory transduction in the context of the orch or theory of consciousness. Int J Radiol Radiat Ther; 10(3): 65-72. DOI: 10.15406/ijrrt.2023.10.00358
25. Ruggiero M. GcMAF in radiation therapy: identification and molecular characterization of the human GcMAF receptor. Int J Radiol Radiat Ther. 2024; 11(3): 77-80. DOI: 10.15406/ijrrt.2024.11.00390
26. Ruggiero M. Chanting of Nam-Myoho-Renge-Kyo in the context of the Buddhist liturgy of Nichiren Shoshu: study of sound frequencies, brain activity, and microbial metabolism. J Neurol Stroke. 2024; 14(4):103-109. DOI: 10.15406/jnsk.2024.14.00592
27. Ruggiero M. Investigating a novel psychoneuroimmunological pathway: natural radioactivity as an external correlate of chanting Nam-Myoho-Renge-Kyo. Int J Radiol Radiat Ther. 2025; 12(5): 120-128. DOI: 10.15406/ijrrt.2025.12.00434
28. Ruggiero, M. & Hiratsuka, A. (2024). On the Natural Intelligence and Consciousness of the Immune System and its Relationship with the Brain. American Journal of Immunology, 19(1), 11-23.
29. Ruggiero, M. (2026). From Signal Transduction to Quantum Biology: The Evolution of a Scientific Paradigm across Thermodynamics, Immunotherapy, and the Study of Consciousness (1908-2026). Zenodo. https: //doi. org/10.5281/zenodo.18237385

### BACKGROUND OF THE INVENTION

The present invention relates to novel acetylated peptides exhibiting immunomodulatory activities, that is Gc protein-derived macrophage activating factor (GcMAF)-like activities.

Human plasma contains a multifunctional protein known as group-specific component (Gc protein) or Vitamin D-binding protein, which exists in several common variants, including Gc1F, Gc1S, and Gc2. The Gcl variants are typically characterized by an O-linked trisaccharide (N-acetylgalactosamine (GalNAc)-galactose-sialic acid) attached at the Threonine residue at position 420(Thr 420 ). Specific enzymatic removal of the terminal sialic acid and galactose residues from this sugar chain converts the Gc1 protein into a potent macrophage activating factor, commonly referred to as GcMAF, which retains the GalNAc moiety linked to Thr420 (Gregory et al. 2010; Nabeshima et al. 2020).

Interestingly, the Gc2 variant, which possesses a Lysine residue instead of Threonine at position 420(T 420 K) and consequently lacks this glycosylation site entirely, has also been shown to activate macrophages in a manner functionally superimposable to that of canonically derived GcMAF (Nabeshima et al. 2020). This observation suggests that the biological activity associated with GcMAF is not solely dependent on the presence of the GalNAc-Thr420 structure.

GcMAF, referring to the activity associated with these Gc protein derivatives, has garnered significant interest as a potential therapeutic agent for a wide array of conditions. These include applications in cancer immunotherapy and anti-angiogenesis, potential treatments for autism spectrum disorders, mitigation of neurological side effects from cancer chemotherapy, and even potential roles in addressing conditions like COVID-19 (US20170173115A1; Nobuto Yamamoto). Furthermore, GcMAF activity shows promise in combination with radiation therapy, potentially enhancing the immune response against residual cancer cells, inhibiting cancer cell proliferation and angiogenesis, mitigating radiation-induced immunosuppression, and minimizing neurological side effects (Ruggiero 2024a). The combination of radiation and immunotherapy is an area of active research, aiming to leverage radiation's ability to induce systemic anti-tumor immunity.

Despite the therapeutic potential, a complete understanding of the molecular mechanisms underlying GcMAF activity, particularly the identification and characterization of its cellular receptor(s), remains crucial but incomplete. Several hypotheses regarding the GcMAF receptor exist. Early work suggested potential interactions with the Vitamin D Receptor (VDR), based partly on structural considerations and correlations with VDR polymorphisms, although this remains debated (US20160220587A1; Marco Ruggiero). More recently, based on the ability of the receptor to bind both the GalNAc-containing GcMAF and the non-glycosylated Gc2 variant, the chemokine receptor CCR1 has been proposed as a candidate receptor (interpreting Nabeshima et al. 2020). Specifically, the first extracellular domain of CCR 1, containing a unique sequence (TTEDYDTTT) rich in negatively charged and Threonine residues, has been suggested as a potential binding site capable of interacting with both the Lysine (of Gc2) and the GalNAc moiety (of GcMAF) through electrostatic interactions, hydrogen bonding, and potentially other non-covalent forces. Another hypothesis involves CLEC 10A (Macrophage lectin 2), which binds GalNAc, though this interaction faces challenges in explaining Gc2 activity and the broader biological effects of GcMAF. The possibility of multiple receptors mediating GcMAF's pleiotropic effects cannot be ruled out.

The finding that Gc2 exhibits GcMAF activity, combined with observations that other molecules like certain sulfated polysaccharides (e.g., chondroitin sulfate, fucoidan) may possess similar activities or interfere with GcMAF effects (potentially via CCR1 binding, as suggested for heparin), lends support to the concept that “GcMAF” might represent a specific type of biological activity rather than being exclusively embodied by a single, defined molecular structure (i.e., Gc protein with GalNAc at Thr420).

Therefore, while GcMAF activity holds significant therapeutic promise and research continues into its natural forms and receptors, there is complexity surrounding the precise molecular entities responsible for the activity and their mechanisms of action (Ruggiero 2024b). This highlights a need in the art for well-defined, synthetic molecules capable of reliably eliciting GcMAF-like biological responses, such as macrophage activation and potential anti-cancer effects, which can be produced with high purity and potentially tailored properties. The present invention addresses this need by providing novel acetylated peptides designed to exhibit such GcMAF-like activity.

### DESCRIPTION OF THE INVENTION

The present invention encompasses two distinct preferred embodiments for the production of novel acetylated peptides for immune modulation, specifically two sequences exhibiting GcMAF-like activity.

The acetyl group (Ac-) at the N-terminus of each peptide serve several important functions:
Protection from N-terminal Degradation: The N-terminus of a peptide contains a free amino group, which can be a target for aminopeptidases, enzymes that cleave amino acids from the N-terminus. The acetyl group blocks this free amino group, making the peptide more resistant to enzymatic degradation and potentially increasing its half-life in biological systems. This is a very common reason for N-terminal acetylation in synthetic peptides intended for therapeutic or research purposes.
Increased Stability: N-terminal acetylation contributes to the overall conformational stability of the peptide.
Influence on Conformation and Folding: The addition of the acetyl group can slightly alter the charge distribution at the N-terminus of the peptide. This can influence the local electrostatic interactions and potentially affect the peptide's folding and secondary structure.

Modulation of Biological Activity: The presence of the acetyl group can affect the peptide's interaction with its target molecules, such as receptors or enzymes. It might enhance binding affinity or efficacy depending on the specific interaction. In the context of GcMAF-like activities, the acetylation is crucial for the peptide to properly interact with macrophages or other components of the immune system.
Mimicking Natural Modifications: Many naturally occurring proteins and peptides are N-terminally acetylated. This modification is often essential for their proper function, localization, or interaction with other cellular components. Acetylating a synthetic peptide might be a strategy to mimic a naturally occurring motif or to enhance its biological relevance.
Reduced Immunogenicity: In some instances, modifications like acetylation can reduce the potential for a peptide to elicit an unwanted immune response when administered in vivo.

In the context of the disclosed invention, the N-terminal acetylation of these novel peptides is likely crucial for their stability and their ability to exert the desired immune modulating activities (GcMAF-like). It could be essential for the peptides to remain intact long enough to interact with the target cells and trigger the intended biological effects.

The novel peptides that are the object of the present invention possess several key characteristics commonly associated with intrinsically disordered domains (IDD). They have a high proportion of charged residues, a relatively low proportion of hydrophobic residues, and a net positive charge. Therefore, they exhibit significant conformational flexibility and lack a well-defined, stable tertiary structure in solution. Therefore, exhibiting features of IDD can confer several biological and pharmacological advantages, particularly for peptides and proteins involved in regulation and signaling:

### Biological Advantages

Enhanced Binding Promiscuity: IDDs can interact with multiple different binding partners. Their flexibility allows them to adopt various conformations to fit different target surfaces, enabling a single protein to participate in diverse cellular processes and pathways.

Molecular Recognition with High Specificity but Low Affinity: IDDs often contain short linear motifs (SLiMs) that can bind to specific folded domains. The low affinity of these interactions allows for rapid association and dissociation, which is crucial for dynamic regulation and signaling.
Scaffolding and Hub Formation: The extended and flexible nature of IDDs allows them to act as scaffolds, bringing together multiple interacting proteins to form large complexes or signaling platforms. They can serve as central hubs in protein interaction networks.
Regulation through Conformational Entropy: The high conformational entropy of IDDs can be modulated upon binding or modification, providing a mechanism for regulating protein activity and interactions. For example, binding can induce folding in an IDD, leading to a functional switch.
Increased Accessibility for Post-translational Modifications (PTMs): The unfolded nature of IDDs makes them more accessible to modifying enzymes (kinases, phosphatases, acetyltransferases, etc.). PTMs can significantly alter the charge, structure, and interaction properties of IDDs, providing a versatile regulatory layer.
Entropic Chains and Linkers: IDDs can function as flexible linkers between folded domains within a protein, allowing these domains to move independently and explore a larger conformational space. This can be important for enzymatic activity or protein complex assembly.
Phase Separation: IDDs are often key drivers of liquid-liquid phase separation (LLPS), leading to the formation of membraneless organelles within the cell. This compartmentalization allows for the concentration of specific molecules and the regulation of biochemical reactions.
Adaptability to Cellular Conditions: The lack of a rigid structure allows IDDs to be more adaptable to changes in cellular environment, such as pH, ion concentration, or the presence of other molecules.
Pharmacological Advantages for therapeutic peptides with IDD features such as those herein disclosed:
Enhanced Cell Penetration: The flexibility can facilitate their passage through cell membranes compared to rigid, folded structures, although this is highly sequence-dependent.
Targeting Protein-Protein Interactions (PPIs): IDDs are often involved in crucial PPIs that are implicated in various diseases. Peptides mimicking or targeting these disordered regions can be designed to disrupt these interactions, offering therapeutic potential.
Increased Bioavailability and Reduced Degradation: While not a universal feature, certain amino acid compositions found in IDDs can confer some resistance to proteolytic degradation, potentially increasing the bioavailability of a therapeutic peptide. N-terminal acetylation is a common strategy to enhance stability.
Design of Conformational Switches: Therapeutic peptides with IDD features can be designed to undergo specific conformational changes upon binding to a target molecule or in response to a specific environment (e.g., tumor microenvironment), leading to targeted drug delivery or activation.
Mimicking Natural Regulators: Peptides that mimic naturally occurring IDDs involved in regulatory processes can be developed as agonists or antagonists to modulate specific pathways.
Potential for Multi-Targeting: The ability of IDDs to interact with multiple partners could be exploited to develop peptides with multi-targeting capabilities, which can be advantageous in complex diseases like cancer.

Due to their relatively small size, the novel acetylated peptides disclosed herein, including those exemplified in the preferred embodiments, are efficiently and cost-effectively produced via chemical synthesis rather than recombinant technology.

### EXAMPLES OF THE PREFERRED EMBODIMENTS

Detailed embodiments of the present invention are disclosed herein, however, it is to be understood that the disclosed embodiments are merely exemplary of the invention, which may be embodied in various forms. Therefore, specific functional details disclosed herein are not to be interpreted as limiting, but merely as a basis for the claims and as a representative basis for teaching one skilled in the art to variously employ the present invention in virtually any appropriately detailed embodiment.

### Example #1: a Novel Acetylated Peptide Endowed with GcMAF-Like Activity

The sequence of the acetylated peptide of Example #1 is Ac-TPTELAKLVNKHSD (Ac-Thr-Pro-Thr-Glu-Leu-Ala-Lys-Leu-Val-Asn-Lys-His-Ser-Asp). The Molecular Weight (MW) is approximately 1597.87 Da.

### Example #2: A Novel Acetylated Peptide Endowed with GcMAF-Like Activity

The sequence of the acetylated peptide of Example #2 is Ac-TPKELAKLVNKRSE (Ac-Thr-Pro-Lys-Glu-Leu-Ala-Lys-Leu-Val-Asn-Lys-Arg-Ser-Glu). The Molecular Weight (MW) is approximately 1636.90 Da.

### Example #3: A Method for the Synthesis of the Two Acetylated Peptides of Examples 1-2 Using the Standard Fmoc/tBu Solid-Phase Peptide Synthesis (SPPS) Strategy

This method involves building the peptide chain step-by-step on a solid resin support, using temporary N-terminal protection (Fmoc) and permanent side-chain protection (acid-labile groups like tBu, Boc, Trt, Pbf), followed by N-terminal acetylation, cleavage from the resin, and purification. Target Peptides include Peptide 1: Ac-TPTELAKLVNKHSD (14-mer); and Peptide 2: Ac-TPKELAKLVNKRSE (14-mer)

Detailed synthesis steps include Step 1: Resin Preparation and First Amino Acid Loading For Peptide 1: Use Wang resin pre-loaded with Fmoc-Asp(OtBu)-OH. For Peptide 2: Use Wang resin pre-loaded with Fmoc-Glu(OtBu)-OH. Step 2 involves peptide chain elongation (iterative cycle) The cycle is repeated for each amino acid, working from the C-terminus to the N-terminus.

For Peptide 1, after loading Asp(OtBu), the sequence of coupling is: Ser(tBu)-His-(Trt)-Lys(Boc)-Asn(Trt)-Val-Leu-Ala-Lys(Boc)-Leu-Glu(OtBu)-Thr(tBu)Pro-Thr(tBu). For Peptide 2, after loading Glu(OtBu), the sequence of coupling is: Ser(tBu)-Arg(Pbf)-Lys(Boc)-Asn(Trt)-Val-Leu-Ala-Lys(Boc)-Leu-Glu(OtBu)-Lys(Boc)-Pro-Thr(tBu).

Step 3 consists of N-Terminal Acetylation after the final Fmoc deprotection. An acetylation solution containing acetic anhydride and DIPEA in DMF is added to the resin and shaken for 30-60 minutes. Step 4 involves cleavage and side-chain deprotection using a cocktail of TFA/TIS/H 2O (95:2.5:2.5 v/v/v) for 2-4 hours. This simultaneously removes all side-chain protecting groups.

Step 5: Characterization and Calculated Monoisotopic Masses [M+H]+. Peptide 1 (Ac-TPTELAKLVNKHSD) has a calculated mass of approximately 1597.87 Da, and Peptide 2 (Ac-TPKELAKLVNKRSE) has a calculated mass of approximately 1636.90 Da.

What is claimed is:

1. An isolated peptide comprising the amino acid sequence Ac-TPTELAKLVNKHSD.

2. The isolated peptide of claim 1, wherein the peptide exhibits GcMAF-like activity.

3. An isolated peptide comprising the amino acid sequence Ac-TPKELAKLVNKRSE.

4. The isolated peptide of claim 3, wherein the peptide exhibits GcMAF-like activity.

5. A method for the synthesis of a peptide of claim 1, comprising the steps of Fmoc/tBu Solid-Phase Peptide Synthesis (SPPS) on a solid resin support, followed by N-terminal acetylation, and cleavage from the resin.

6. A method for the synthesis of a peptide of claim 3, comprising the steps of Fmoc/tBu Solid-Phase Peptide Synthesis (SPPS) on a solid resin support, followed by N-terminal acetylation, and cleavage from the resin.
